(19) United States
(12) Patent Application Publication
(10) Pub. No.: US 2023/0010415 A1
(43) Pub. Date: Jan. 12, 2023

(54) CRANIOFACIAL IMPLANT INCLUDING A PASSIVE PRESSURE SENSOR

(71) Applicant: Longeviti Neuro Solutions LLC, Hunt Valley, MD (US)

(72) Inventors: Jesse Christopher, Hunt Valley, MD (US); Bradley Rabinovitz, Severna Park, MD (US)

(21) Appl. No.: 17/811,241

(22) Filed: Jul. 7, 2022

Related U.S. Application Data
(60) Provisional application No. 63/219,232, filed on Jul. 7, 2021.

(51) Int. Cl. A61F 2/28; A61B 5/03; A61B 8/00

(52) U.S. Cl. CPC A61F 2/2875; A61B 5/031; A61B 8/4416; A61B 2562/0247

(57) ABSTRACT

A craniofacial implant includes a craniofacial implant body and a passive pressure sensor. The craniofacial implant body permits measurement of the passive pressure sensor via externally applied stimuli passing through the craniofacial implant body.

### CROSS REFERENCE TO RELATED APPLICATION

This application claims the benefit of U.S. Provisional Patent Application Ser. No. 63/219,232, entitled “CRANIOFACIAL IMPLANT INCLUDING A PASSIVE PRESSURE SENSOR,” filed Jul. 7, 2021, which is incorporated herein by reference.

### BACKGROUND OF THE INVENTION

### 1. Field of the Invention

The present invention relates to a craniofacial implant including a passive pressure sensor.

### 2. Description of the Related Art

Hydrocephalus is a condition in which an excessive accumulation of cerebral spinal fluid is encountered. Cerebral spinal fluid is the clear fluid that surrounds the brain and the spinal cord. The excessive accumulation results in abnormal dilation of the ventricles within the brain. This dilation may cause the accumulation of potentially harmful pressure on the tissues of the brain.

It is, therefore, important to monitor the pressure within cranium.

### SUMMARY

In one aspect a craniofacial implant includes a craniofacial implant body and a passive pressure sensor. The craniofacial implant body permits measurement of the passive pressure sensor via externally applied stimuli passing through the craniofacial implant body.

In some embodiments the craniofacial implant body is sonolucent.

In some embodiments the externally applied stimuli is ultrasound.

In some embodiments the craniofacial implant body includes an outer surface, an inner surface, and a peripheral edge shaped and dimensioned for engagement with a skull of a patient upon implantation, and the passive pressure sensor is positioned adjacent the inner surface such that the passive pressure sensor is exposed to intracranial fluid for sensing intracranial pressure.

In some embodiments the craniofacial implant body is sonolucent and the externally applied stimuli is ultrasound.

In some embodiments the craniofacial implant body includes at least one cavity accommodating displacement of the passive pressure sensor.

In some embodiments the craniofacial implant body includes structure holding the passive pressure sensor in place.

In some embodiments a recess is formed along the inner surface of the craniofacial implant body and the passive pressure sensor is positioned within the recess.

In some embodiments the craniofacial implant body is comprised of clear PMMA (Poly(methyl methacrylate).

In some embodiments the craniofacial implant body is sonolucent and the craniofacial implant body has low sound loss permitting measurement of the passive pressure sensor with ultrasonic imaging.

In some embodiments the craniofacial implant body is sonolucent and an ultrasound transducer is calibrated or initialized with the passive pressure sensor and/or the craniofacial implant body.

In some embodiments the craniofacial implant body is sonolucent and the craniofacial implant further includes an ultrasound transducer transmitting sound waves for interaction with the passive pressure sensor in a predetermined manner based upon physical characteristics of the passive pressure sensor.

In some embodiments a plurality of a passive pressure sensors are provided.

In some embodiments the passive pressure sensor is integrally constructed with the craniofacial implant body.

In some embodiments a mounting plate in which the craniofacial implant body is selectively positioned is provided.

In some embodiments the mounting plate include a hollowed-out center aperture shaped and dimensioned for placement and mounting of the craniofacial implant body therein.

In some embodiments the mounting plate comprises PMMA (Poly(methyl methacrylate), PEEK (Polyether ether ketone), PEKK (Polyetherketoneketone), porous polyethylene, and/or other tissue-engineered constructs.

In some embodiments the craniofacial implant body is sonolucent and the craniofacial implant body includes an alignment feature aiding alignment with an external ultrasound transducer.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a perspective view of a craniofacial implant in accordance with a first embodiment.

FIG. 2 is an exploded view of the craniofacial implant shown in FIG. 1.

FIG. 3 is a cross sectional view of the craniofacial implant shown in FIG. 1.

FIG. 4 is a perspective view of a craniofacial implant in accordance with a second embodiment.

FIG. 5 is an exploded view of the craniofacial implant shown in FIG. 4.

FIG. 6 is a cross sectional view of the craniofacial implant shown in FIG. 4.

FIGS. 7 and 8 show an illustration of the assembly of an illustrative microfluidics passive pressure sensing device.

FIG. 9 is a perspective view of a craniofacial implant in accordance with a third embodiment.

FIG. 10 is a cross sectional view of the craniofacial implant shown in FIG. 9.

FIG. 11 is a perspective view of a craniofacial implant in accordance with a fourth embodiment.

FIG. 12 is a cross sectional view of the craniofacial implant shown in FIG. 11.

FIG. 13 is a perspective view of an alignment feature.

FIG. 14 shows front and back perspective views of a craniofacial implant in accordance with a non-custom embodiment.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

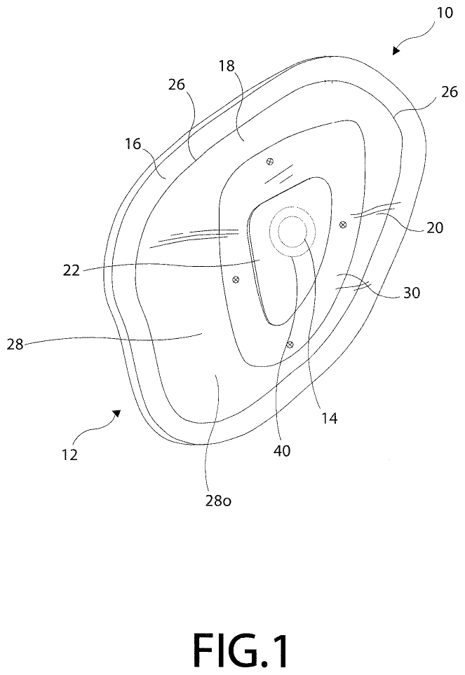

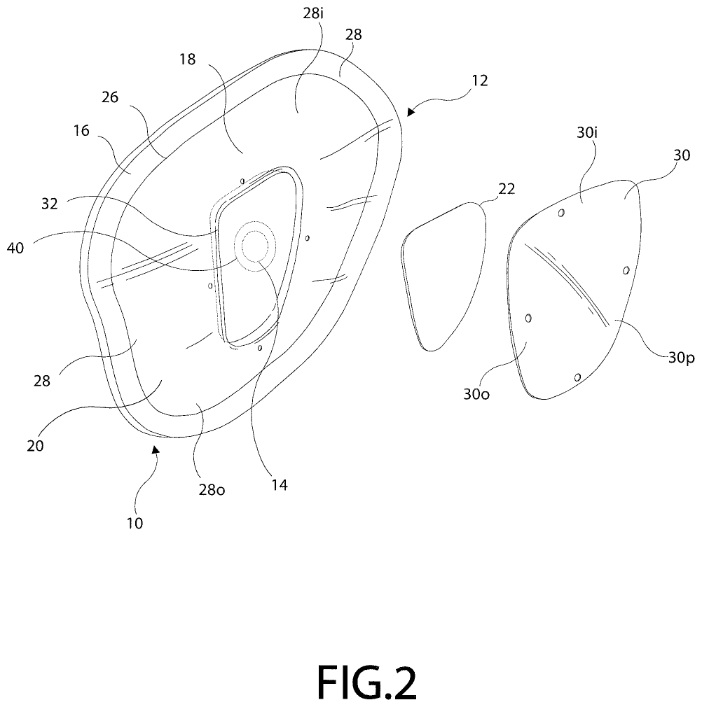

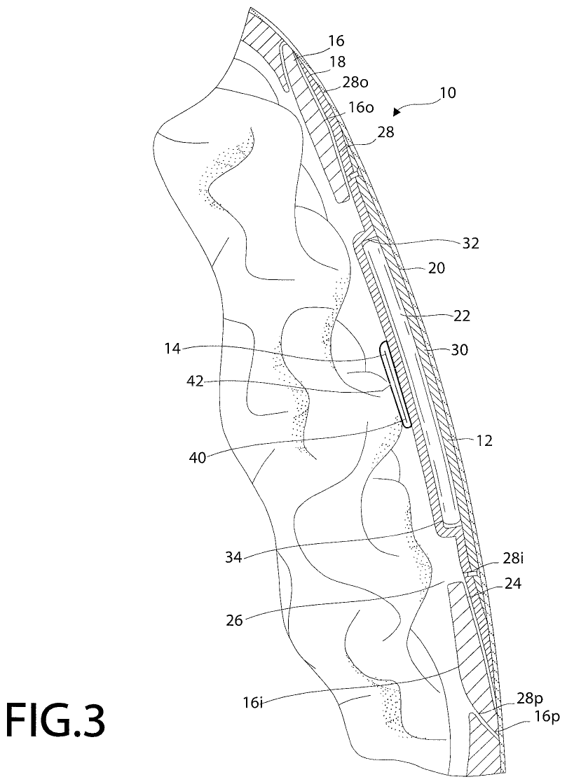

Referring to FIGS. 1, 2, and 3, a craniofacial implant 10 is disclosed. Generally, the craniofacial implant 10 includes a sonolucent craniofacial implant assembly 12 and a passive pressure sensor 14 allowing for the measurement of intracranial pressure. The present craniofacial implant 10 takes advantage of the combination of a the lucent characteristics of the material of the sonolucent craniofacial implant assembly 12 and the structural stability offered by the sonolucent craniofacial implant assembly 12 as it replaces a resected portion of a patient's skull.

As used in accordance with the present disclosure, the term “passive” is used to distinguish the pressure sensor of the present invention from those sensors that actively gather and transmit data for use in identifying specific operating parameters. Passive sensors of various forms are known in the art, and this term is intended to be utilized in accordance with the present disclosure in the manner understood by those skilled in the art. As such, passive pressure sensors in accordance with the present disclosure form part of remote sensing systems measuring naturally occurring changes in the physical characteristics of an object or material (that is, the passive sensor) without the direct application of power to the object or material and based upon the sensed alteration of externally applied and transmitted signals, energy, etc. by a remote sensing device. As such, passive pressure sensors of the present disclosure alter externally applied stimuli (for example, sound waves, light, etc.) passing through an implant body to produce an output that is discerned remotely for the determination of parameters associated with changes in the environment of the passive sensor. Examples of passive sensing technologies include, but are not limited to, sound based (for example, ultrasound), light (both visible and nonvisible), thermal, electric field sensing, chemical, infrared, and seismic.

The craniofacial implant assembly 12 of the present invention may be of the type described in International Patent Application PCT/US2016/030447, filed May 2, 2017, entitled “LOW PROFILE INTERCRANIAL DEVICE,” (published as WO 2017/039762), U.S. patent application Ser. No. 15/669,268, filed Aug. 4, 2017, entitled “METHOD FOR MANUFACTURING A LOW-PROFILE INTERCRANIAL DEVICE AND THE LOW-PROFILE INTERCRANIAL DEVICE MANUFACTURED THEREBY” (published as U.S. Patent Application Publication No. 2018/0055640), and U.S. patent application Ser. No. 16/203,357, filed Nov. 28, 2018, entitled “UNIVERSAL LOW-PROFILE INTERCRANIAL ASSEMBLY” (published as U.S. Patent Application Publication No. 2019/0209328, '328 Publication) all of which are incorporated herein by reference.

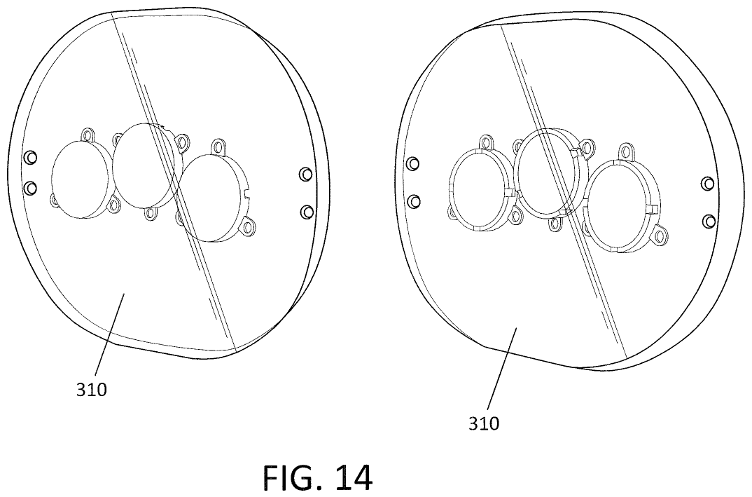

While an embodiment of a specific craniofacial implant assembly, which includes a custom implant, is disclosed with reference to FIGS. 1, 2, and 3, it is appreciated the implant 310 may be of a non-custom construction as disclosed for example with reference to FIG. 14. Such an embodiment would include the functional features of the implants disclosed herein, but would be manufactured in a manner that is not specific to a patient and as such the resected portion of the skull would necessarily be shaped to match the implant 310 of FIG. 14.

For the purposes of describing the craniofacial implant assembly 12 herein it will be presumed the craniofacial implant assembly 12 is a universal low-profile intercranial assembly as disclosed in the '328 Publication referenced above. The universal low-profile intercranial assembly 12 is generally composed of mounting plate 16 into which a low profile intercranial device 18 is mounted. The low profile intercranial device 18 is composed of a static craniofacial implant body 20 and a functional neurosurgical implant 22 (for example, an ultrasound transducer for use in conjunction with the passive pressure sensor 14 where the passive pressure sensor is a hydrogel pressure sensor as discussed below). This combination of elements results in the present universal low-profile intercranial assembly 12 that provides a mechanism whereby various low profile intercranial devices 18 may be implanted as desired and needed based upon the progress of a patient undergoing cranial and/or brain-based treatments.

The static craniofacial implant body 20 is a prefabricated implant manufactured from clear poly (methyl methacrylate) (PMMA) or any other clear biocompatible material suited for safe use in craniofacial reconstruction. While a clear PMMA static craniofacial implant body 20 is used in accordance with a preferred embodiment as discussed herein, it is appreciated the prefabricated clear static craniofacial implant body 20 may include a polymer, metal, bioengineered material, or any combinations thereof. For example, the prefabricated clear static craniofacial implant body 20 may include any biomaterial that may allow enhanced visibility with complete translucency. In addition, it is appreciated the use of the term static craniofacial implant body 20 herein is intended to include all clear implants that may be used in conjunction with skull reconstruction procedures, facial reconstruction, or any combination thereof. As used herein the term “clear” is intended to refer to a material that is substantially completely transparent (for example, the static craniofacial implant body 20 is transparent with the exception of a neurological device(s) that might be integrated into the static craniofacial implant body 20 and which does not otherwise impede the ability to achieve the underlying principles of the invention) and exhibits the property of transmitting rays of light through its substance so that bodies situated beyond or behind can be distinctly seen when looking through the material.

The clear static craniofacial implant body 20 is also sonolucent (that is, allowing passage of ultrasonic waves without production of echoes that are due to reflection of some of the waves). As a result, the clear static craniofacial implant body 20 is composed of clear sonolucent PMMA that allows for both intraoperative and postoperative trans-cranioplasty ultrasound. The static craniofacial implant body 20 has low sound loss permitting measurement of the hydrogel pressure sensor 104 with ultrasonic imaging; that is, the clear static craniofacial implant body 20 also exhibits attenuation characteristics resulting in minimal degradation of the ultrasonic waves generated by the transducer 22 of an ultrasound system. In accordance with a disclosed embodiment, the clear craniofacial implant body 20 has a thickness ranging between 3.0 mm-6.5 mm with a mean thickness of 5.4 mm, which is consistent with native bone flap thickness. While clear sonolucent PMMA is disclosed in accordance with a preferred embodiment, it is appreciated other materials, for example, clear sonolucent PEEK, may be used. Further to the interaction of ultrasound with the hydrogel pressure sensor 14, the implementation of ultrasound allows for intraoperative trans-cranioplasty ultrasound visualization, for example, of recognizable ventricular anatomy. Furthermore, postoperative bedside trans-cranioplasty ultrasound allows for visualization, for example, of comparable ventricular anatomy and a small epidural fluid collection corresponding to that visualized on an axial computed tomography (CT) scan. Accordingly, the present clear craniofacial implant body 20 with sonolucent characteristics offers great promise for enhanced diagnostic and therapeutic applications previously limited by cranial bone. Furthermore, the present clear craniofacial implant body 20 with sonolucent characteristics allows for the possibility of housing implantable devices to provide for real-time surveillance of intracranial pathology.

The static craniofacial implant body 20 may also be radiolucent (that is, allowing passage of radio waves without production of echoes that are due to reflection of some of the waves).

By way of example, the clear static craniofacial implant body 20 may be manufactured in a manner allowing for the transmission of ultrasonic waves as described in U.S. Pat. No. 9,044,195, entitled “IMPLANTABLE SONIC WINDOW” ('195 Patent) which is incorporated herein by reference. As explained in the '195 Patent, a strong, porous sonically translucent material through which ultrasonic waves can pass for purposes of imaging the brain is employed, wherein the material is a polymeric material, such as polyethylene, polystyrene, acrylic, or poly(methyl methacrylate) (PMMA). In addition, U.S. Pat. No. 9,535,192, entitled “METHOD OF MAKING WAVEGUIDE-LIKE STRUCTURES” ('192 Publication) and U.S.

Patent Application Publication No. 2017/0156596, entitled “CRANIAL IMPLANTS FOR LASER IMAGING AND THERAPY” ('596 Publication) both of which are incorporated herein by reference, making waveguide-like structures within optically transparent materials using femtosecond laser pulses wherein the optically transparent materials are expressly used in the manufacture of cranial implants. The '596 Publication explains the use of optically transparent cranial implants and procedures using the implants for the delivery of laser light into shallow and/or deep brain tissue. The administration of the laser light can be used on demand, thus allowing real-time and highly precise visualization and treatment of various pathologies. Further still, Tobias et al. describe an ultrasound window to perform scanned, focused ultrasound hyperthermia treatments of brain tumors. Tobias et al., “ULTRASOUND WINDOW TO PERFORM SCANNED, FOCUSED ULTRASOUND HYPERTHERMIA TREATMENTS OF BRAIN TUMORS,” Med. Phys. 14(2), March/April 1987, 228-234, which is incorporated herein by reference. Tobias et al. tested various materials to determine which material would best serve as an acoustical window in the skull and ultimately determined polyethylene transmitted a larger percentage of power than other plastics and would likely function well as an ultrasonic window. Further still, Fuller et al., “REAL TIME IMAGING WITH THE SONIC WINDOW: A POCKET-SIZED, C-SCAN, MEDICAL ULTRASOUND DEVICE,” IEEE International Ultrasonics Symposium Proceedings, 2009, 196-199, which is incorporated herein by reference, provides further information regarding sonic windows.

Radiolucency as applied to the present invention allows a clinician to see the anatomy surrounding the clear static craniofacial implant body 20 without “scatter” or interfering artifacts from the implant for diagnosis and follow-up. By another definition of radiolucency, radio waves are able to transmit easily through the clear static craniofacial implant body 20, for example, via Bluetooth or other frequency transmission, which can serve many purposes including, but not limited to, data management and controller telemetry. The provision of radiolucency also allows for the integration of markings (as discussed below) made with radiographic materials, for example, barium sulfate, to be visible in contrast to the remainder of the cranial implant body to allow for unique device identifiers or unique patient information to be visible on post-operative scans.

Considering the provision of optical lucency in the present clear static craniofacial implant body 20, the ability to optically transmit through the clear static craniofacial implant body 20 allows for visualization of anatomy distal to the clear static craniofacial implant body 20 (as previously described), allows for the potential of higher bandwidth optical links (similar to radio transmission) between proximal adjunct devices, allows for light to be emitted from the clear static craniofacial implant body 20 to adjacent anatomy which could aid in optogenetics, and allows for imaging/therapeutic modalities that rely on light like optical coherence tomography from within the implant.

The mounting plate 16 and/or the static craniofacial implant body 20 are manufactured. The mounting plate 16 is augmented, reduced and/or modified to include a hollowed-out center aperture 26 shaped and dimensioned for the ready placement and mounting of the low profile intercranial device 18 therein. In this way, and as will appreciated based upon the following disclosure, the mounting plate 16 is specifically shaped and dimensioned for intercranial placement within the cranial defect while simultaneously providing a center aperture 26 into which a low profile intercranial device 18 may be readily mounted. Given that the center aperture 26 of the mounting plate 16 is of a known shape, which may be readily replicated and controlled, the shape of the low profile intercranial device 18 can be readily controlled to allow for immediate and exact placement of the low profile intercranial device 18 within the center aperture 26. This allows for a first low profile intercranial device 18 to be implanted and used at a first stage of a patient's treatment and subsequently removed and replaced with a second low profile intercranial device at a second stage of the patient's treatment.

Considering now the structural details of the mounting plate 16, the mounting plate 16 includes an outer (commonly convex) first surface 16o, an inner (commonly concave) second surface 16i, and a peripheral edge 16p extending between the outer first surface 16o and the inner second surface 16i. The mounting plate 16 is shaped and dimensioned for engagement with the skull of the patient upon implantation in a manner well known to those skilled in the field of neurosurgical procedures. The outer first surface 16o and inner second surface 16i of the mounting plate 16 are preferably curved in a superior to inferior direction, a posterior to anterior direction, and a medial to lateral direction. In addition, and as noted in the embodiments discussed with reference to FIGS. 1, 2, and 3 the peripheral edge 16p has a substantial taper for resting upon a matching taper formed along the skull. It is, however, appreciated that this taper may vary (or not exist at all, that is, the peripheral edge 16p may be substantially perpendicular relative to the outer first surface 16o and the inner second surface 16i) depending upon the specific needs of the procedure. In accordance with a preferred embodiment, the mounting plate 16 will have a preselected thickness not exceeding the space between the inner surface of the scalp and the outer surface of the dura, for example, in the range of around 1 millimeter to 25 millimeters (with areas of strategic bulking and/or thinning) and depending upon the strength of the materials used in the construction of the mounting plate 16. Preferably, the mounting plate 16 will have a thickness of 1 millimeter to 12 millimeters. As mentioned above, the mounting plate 16 also includes a center aperture 26 designed to accommodate the static craniofacial implant body 20. The center aperture 26 is defined by an inner wall 24 extending between outer first surface 16o and inner second surface 16i of the mounting plate 16.

Considering the static craniofacial implant body 20, it should first be appreciated, the term “static” is used in the description of the present invention because the static craniofacial implant body 20, has no encapsulated inner working (i.e., “functional”) parts, batteries, wires, or computers, and is essentially an improved “empty-shell” which optimizes the inter-implant positioning within the confines of the skull and the neighboring functional neurosurgical implant 22. Such implants may take a variety of forms and are most commonly shaped and dimensioned for integration into the structure of a patient's skull; that is, the static craniofacial implant body 20 has a geometry that substantially conforms to a resected portion of the patient's anatomy to which the implant is to be secured.

In accordance with a disclosed embodiment, the static craniofacial implant body 20 has a two-piece construction allowing for ready access to the functional neurosurgical implant 22 without the need for complete removal of the low-profile intercranial device. The two-piece static craniofacial implant body 20 has no encapsulated inner working parts, batteries, wires, or computers, and is essentially an improved “empty-shell.”

The two-piece static craniofacial implant body 20 in accordance with this embodiment includes a base cranial implant member 28 and a cover cranial implant member 30. The base cranial implant member 28 has a geometry that substantially conforms to a resected portion of the patient's anatomy to which the low-profile intercranial device is to be secured. The base cranial implant member 28 includes an outer (commonly convex) first surface 28o, an inner (commonly concave) second surface 28i, and a peripheral edge 28p extending between the outer first surface 28o and the inner second surface 28i. The static craniofacial implant body 20 is shaped and dimensioned for engagement with the skull of the patient upon implantation in a manner well known to those skilled in the field of neurosurgical procedures. The outer first surface 28o and inner second surface 28i of the base cranial implant member 28 are preferably curved in a superior to inferior direction, a posterior to anterior direction, and a medial to lateral direction.

The base cranial implant member 28 also includes a center recess 32 formed along the outer first surface 28o and optional structural elements, for example, tunnels, channels, pockets, access holes, and/or other structural elements, designed to accommodate various features of the functional neurosurgical implant 22. Multiple recesses may be employed where the functional neurosurgical implant(s) being used dictates and that the recess need not be directly in the center of the base cranial implant member 28 but may be offset as dictated by the procedure being performed.

In addition to the base cranial implant member 28, the two-piece static craniofacial implant body 20 includes a cover cranial implant member 30. The cover cranial implant member 30 is shaped and dimensioned for positioning over the center recess 32 along the outer first surface 28o of the base cranial implant member 28. In accordance with a preferred embodiment, the cover cranial implant member 30 is secured to the base cranial implant member 28 by screw fixation. The cover cranial implant member 30 includes an outer (commonly convex) first surface 30o, an inner (commonly concave) second surface 30i, and a peripheral edge 30p shaped and dimensioned for engagement with the outer first surface 28o of the base cranial implant member 28 along the periphery of the center recess 32. As with the base cranial implant member 28, the outer first surface 30o and inner second surface 30i of the cover cranial implant member 30 are preferably curved in a superior to inferior direction, a posterior to anterior direction, and a medial to lateral direction.

The base cranial implant member 28 and the cover cranial implant member 30 have a total thickness similar to that of the embodiment described above, that is, and depending on the strength characteristics of the materials used, the base cranial implant member 28 and the cover cranial implant member 30 will have a thickness (with areas of strategic bulking and/or thinning) of around 1 millimeter to 25 millimeters, preferably, 1 millimeter to 12 millimeters.

As mentioned above, the cover cranial implant member 30 fits over the center recess 32 along the outer first surface 28o of the base cranial implant member 28. In this way, the inner second surface 30i of the cover cranial implant member 30 and the outer first surface 28o of the base cranial implant member 28, along the center recess 32, define a center cavity 34 configured to conform to the exact requirements of the functional neurosurgical implant 22 in accordance with the present invention. With this in mind, the inner second surface 30i of the cover cranial implant member 30 may be shaped and/or contoured to enhance the positioning of the functional neurosurgical implant 22 within the center cavity 34.

In accordance with one embodiment (as disclosed above), the functional neurosurgical implant of the present invention is preferably an ultrasound transducer 22 for interaction with the passive pressure sensor 14 when the passive pressure sensor is a hydrogel pressure sensor. The ultrasound transducer 22 is calibrated or initialized with the passive pressure sensor 14 and/or the craniofacial implant assembly 12. It should be appreciated that the ultrasound transducer 22 may be integrated with other functional neurosurgical devices, and these other functional neurosurgical device may be integrated into the low profile intercranial device 18 or the additional functional neurosurgical device may be positioned remote from the low profile intercranial device 18.

As briefly discussed above, the craniofacial implant assembly 12, in particular, the base cranial implant member 28 of the static craniofacial implant body 20 includes an outer surface 28o, an inner surface 28i, and a peripheral edge 28p, and the passive pressure sensor 14 is positioned adjacent the inner surface 28i such that the passive pressure sensor 14 is exposed to the intracranial fluid for the purposes of sensing intracranial pressure. In accordance with a disclosed embodiment, a plurality of the hydrogel pressure sensors 14 are used.

In accordance with one embodiment the craniofacial implant assembly 12 includes structure for holding the passive pressure sensor 14 in place. For example, the passive pressure sensor 14 is integrally constructed with the craniofacial implant assembly 12. For example, a cavity 40 is formed adjacent the inner surface 28i of the base cranial implant member 28 of the static craniofacial implant body 20 and the passive pressure sensor 14 is positioned within the cavity. However, the cavity 40 is exposed to the intracranial environment via a hole 42 formed along the inner surface 28i of the cranial implant member 28 that fluidly connects the inner surface 28i and the cavity 40 so that the passive pressure sensor 14 is exposed to the pressure within the intracranial space.

Considering such an embodiment, it is appreciated it would be possible to use a variety of both passive and active pressure sensors.

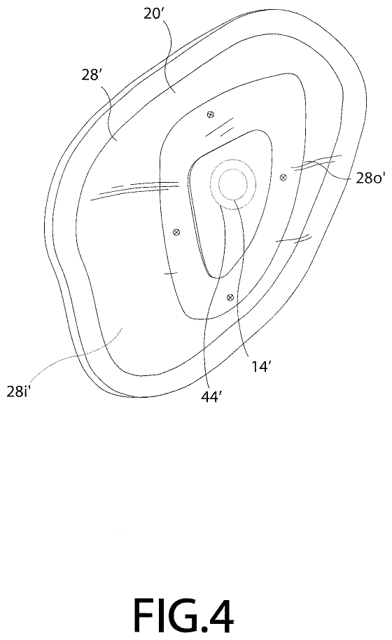

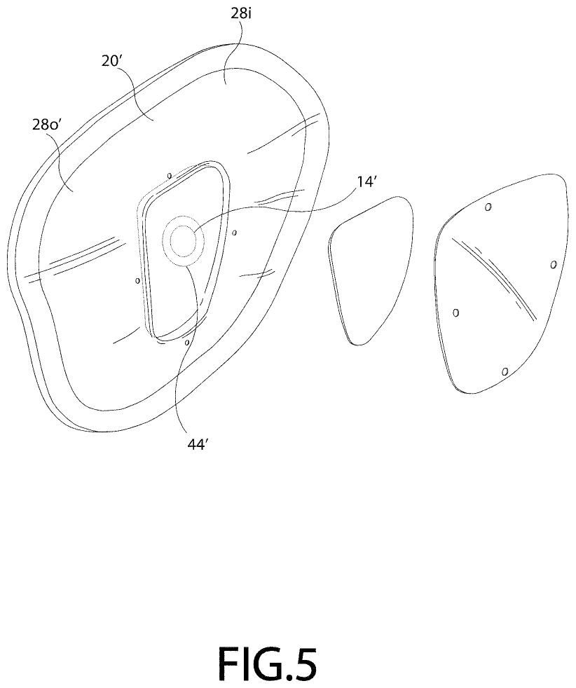

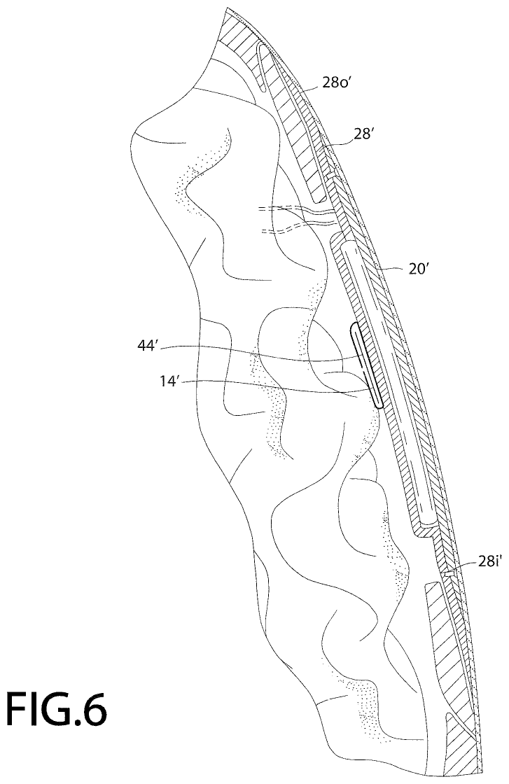

In accordance with another embodiment as disclosed with reference to FIGS. 4, 5, and 6, the passive pressure sensor 14′ is positioned within a recess 44′ formed along the inner surface 28i′ of the base cranial implant member 28′ of the static craniofacial implant body 20′. For example, the recess 44′ if formed along the inner surface 28i′ of the base cranial implant member 28′ of the static craniofacial implant body 20′ and the passive pressure sensor 14′ is positioned within the recess 44′. The passive pressure sensor 14′ is held in place using adhesive or mechanical mounting structures to securely hold the passive pressure sensor 14′ within the recess 44′ and adjacent to the inner surface 28i′ of the base cranial implant member 28′.

In accordance with a disclosed embodiment, the passive pressure sensor 14 is a hydrogel pressure sensor as disclosed in U.S. Patent Application Publication No. 2020/0114353, entitled “Low-Cost Microfluidic Sensors with Smart Hydrogel Patterned Arrays Using Electronic Resistive Channel Sensing for Readout” ('353 Publication), which is incorporated herein by reference. In accordance with the use of the hydrogel pressure sensor as disclosed in the '353 Publication, the craniofacial implant assembly 12 permits the measurement of the passive pressure sensor 14 with ultrasonic imaging. Ultrasound is used, at a specific frequency to which the hydrogel pressure sensor 14 is tuned, to respond to as a passive mechanical resonator and to read out pressure while retaining sonolucent properties. Changes in resonance response are due to applied stress/strain.

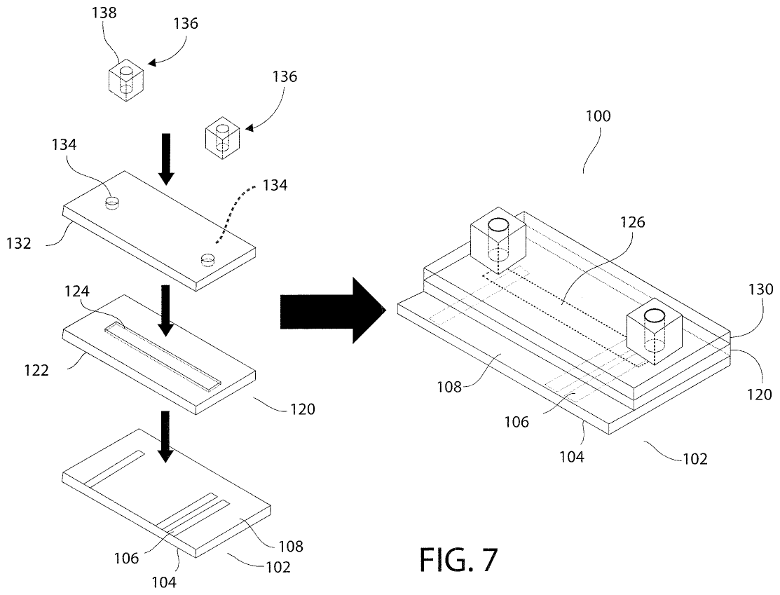

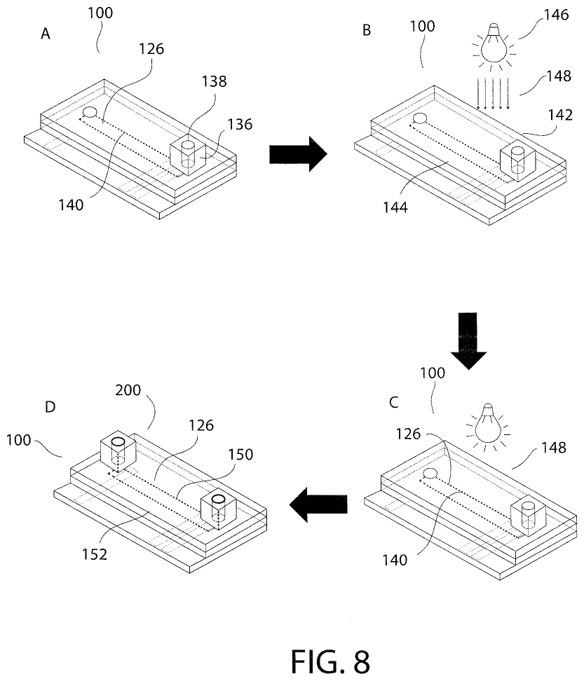

Briefly, FIGS. 7 and 8 shows an illustration of the assembly of an illustrative microfluidics device 100 (for in situ patterning of hydrogel pillars, as described below) used as a pressure sensor in accordance. The microfluidics device 100 can be manufactured using a low-cost fabrication approach with the microfluidic channels fabricated employing a computer controlled cutting plotter.

As explained in the '353 Publication,
. . . the microfluidics device 100 of [FIG. 7] comprises three main layers—a bottom layer 102, a center layer 120, and a top layer 130. The bottom layer 102 in the device 100 comprises a base substrate 104, comprising a (rectangular) piece of polycarbonate (40 mm×75 mm×0.25 mm), with electrodes 106 (e.g., silver paste electrodes) (MG Chemical) (1 mm 25 mm 0.04 mm) stenciled or affixed onto a surface 108 of the base 104. As illustrated in FIG. 7, one electrode 106 or multiple electrodes 106 can be placed at opposing ends of the base 104. The center layer 120 comprises an adhesive film layer 122 (e.g., polyvinyl chloride (PVC) adhesive film) that binds the (bottom and top) layers together and that also serves as the microchannel structure. Specifically, an elongated channel 124 is cut through the adhesive film 122 to form the microchannel 126 in the assembled device 100. The channel 124 and/or microchannel 126 formed thereby can have a length of about 35 mm, a width of about 1.6 mm, and a depth of about 50 μm, in some embodiments. Accordingly, the center (adhesive) layer 120 can have a thickness of about 50 μm, in some embodiments. The top layer 130 comprises a covering 132, comprising another (rectangular) piece of polycarbonate (25 mm×75 mm 0.25 mm), with holes 134 punched or extending therethrough (and serving as inlet/outlet ports to access the microfluidic channel 126 in the assembled device 100). The top layer 130 can be slightly smaller (width wise) than the bottom layer 102 to allow access to the electrodes 106 for measurement. To make interfacing with the device 100 and/or microfluidic channel 126 simple or convenient, connectors 136 (e.g., for attaching microfluidic tubing) can be attached to the top layer 130 over the holes 134. The connectors 136 can comprise a block of PDMS having an access port 138 extending therethrough.
FIG. 8 demonstrates an illustrative method of forming or patterning smart hydrogel features (e.g., an array of smart hydrogel pillars) in a microfluidic channel. For ease of illustration, [FIG. 8] depicts the microfluidics device 100 of [FIG. 7] (modified, as indicated). An array of smart hydrogel features (e.g., an array of distinct, spaced-apart, smart hydrogel pillars extending transverse across the microfluidic channel) can be fabricated inside an enclosed microchannel 126 of the microfluidics device 100 using an in situ photopolymerization technique, described below.
Once the 3-layer microfluidic device 100 is cut and assembled, a pre-gel (fluid) hydrogel solution 140 (described in further detail, below), is introduced (e.g., using capillary forces) into the microchannel 126 via the access port 138 in the connectors 136 and the hole 134 of top layer 130 (or the covering 132 thereof), as shown in [FIG. 8(A)]. Illustratively, a 13 wt % pre-gel (fluid) hydrogel solution containing 80 mol % acrylamide, mol % 3-acrylamidophenylboronic acid, 10 mol % N [3-(dimethylamino)propyl] methacrylamide, 2 mol % N,N′-methylenebisacrylamide and a free-radical photoinitiator can be fluidly introduced into microchannel 126. Illustratively, the smart hydrogels disclosed herein, comprised of 13 wt % of the monomers, were copolymers containing 80 mol % acrylamide from Fisher Scientific (Hampton, N.H., USA), 8 mol % 3-acrylamidophenylboronic acid from Achemo (Hong Kong, China), 10 mol % N-[3-(dimethylamino)propyl] methacrylamide from Polysciences Inc. (Warrington, Fla., USA), and 2 mol % N,N′-methylenebisacrylamide from Sigma-Aldrich (St. Louis, Mo., USA).
Subsequently, as shown in [FIG. 8(B)], a (dark field) photomask 142 having aperture(s) 144 arranged in the desired feature (e.g., pillar array) design is placed over the microchannel 126. Illustratively, the apertures 144 in the phot mask 142 can be round (to form cylindrical pillars) or any other suitable geometric shape (e.g., oval, square, rectangular, etc.). Photo patterning of the array is accomplished by directing collimated UV light 148 from a UV light source 146 through the apertures 144 to polymerize the hydrogel 140 to form (solid or semi-solid) smart hydrogel pillars 150 within the microchannel 126. Illustratively, the hydrogels were polymerized via crosslinking copolymerization using lithium phenyl-2,4,6-trimethylbenzoylphosphinate from Sigma-Aldrich (St. Louis, Mo., USA) as the UV free radical initiator. The light source was a collimated Hg-vapor lamp. While patterning the hydrogel pillars, a dark field chromium photomask with the desired pillars pattern was placed over the channel. Collimated UV light from a mask aligner (Model 206; OAI, San Jose, Calif., USA), with an initial intensity of 13.5 W/cm2 and an exposure time of 5.5 s, was used to polymerize the hydrogel to form pillars within the microchannel.
Illustratively, after this first photo patterning is complete, the mask 142 is removed, as shown in [FIG. 8(C)], and the entire microchannel 126 (containing (unpolymerized) hydrogel pre-gel hydrogel solution 140 and (at least partially polymerized) smart hydrogel pillars 150 (see [FIG. 8(D)]) is flood exposed to the UV light for another quarter of the previous masked exposure time. Illustratively, 1.5 s of UV exposure was flood applied to the channel itself, after the photo patterning was complete and the mask was removed. In certain embodiments, this additional step may be necessary to polymerize a thin hydrogel layer across the channel to enhance adhesion of the hydrogel pillars to the channel and to keep their regular arrangement. Specifically, the shortened, flood exposure process created a thin film of hydrogel between the pillars to keep the pillars from being flushed away during the introduction of analyte solutions. Without being bound to any theory, when this step was not carried out in the current embodiment, it was observed that the patterned pillars did not keep their locations in the channel and were easily flushed out by the surrounding flow. Moreover, the UV light intensity decreased slightly from its initial value at the beginning of the experiments. Hence, the exposure time was adjusted accordingly to ensure a constant exposure dose for all experiments.
The unpolymerized or incompletely polymerized hydrogel (solution) can then be, optionally, flushed or washed from the microchannel by irrigating the channel with a (wash) buffer, solution, or water, leaving only the (polymerized) array 152 of smart hydrogel features (pillars) 150 in the microfluidic channel 126. The resulting device, a microfluidics sensing device 200, comprises the microfluidics device 100 and the array 152 of smart hydrogel features (pillars) 150 in the microfluidic channel 126 thereof. Illustratively, the pillars 150 can be substantially cylindrical and, optionally, regularly spaced apart, due to the configuration (e.g., shape and spacing) of the apertures 144 in the photo mask 142.

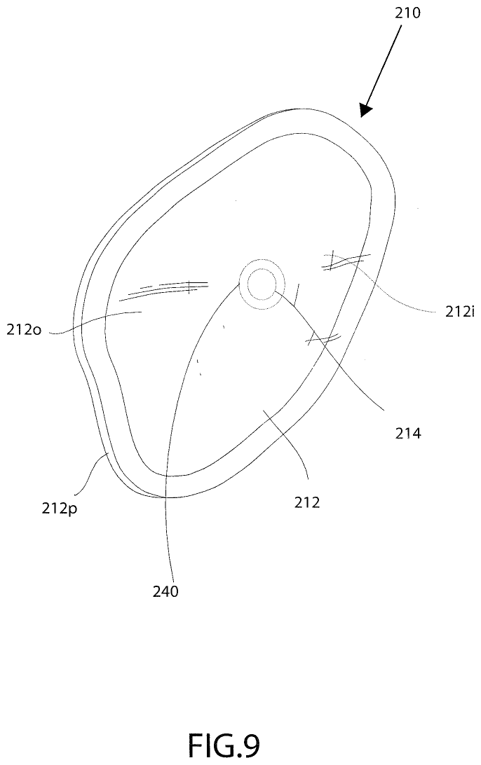

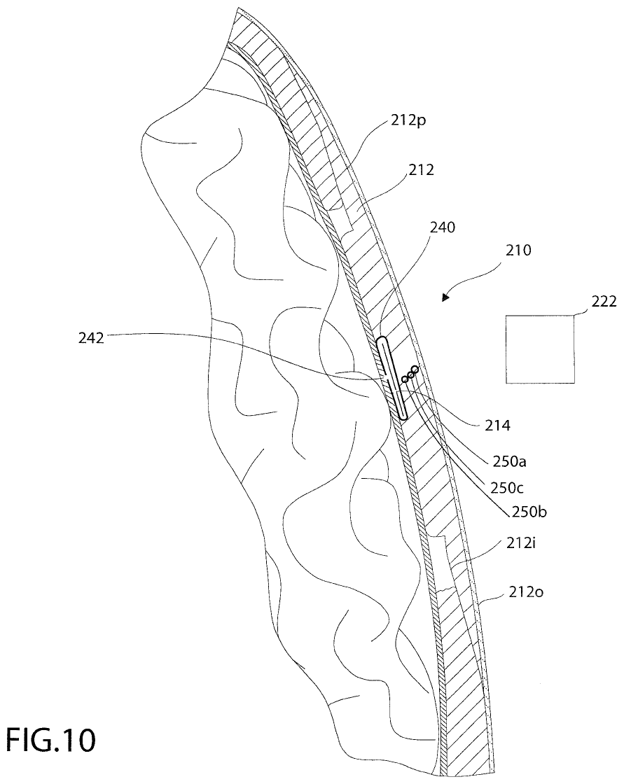

In accordance with another embodiment as shown with reference to FIGS. 9 and 10, the craniofacial implant 210 is constructed such that the ultrasound transducer 222 is not integrated into the sonolucent craniofacial implant assembly 212. Rather, a conventional handheld ultrasound transducer (or other remote monitoring device) is used in conjunction with the craniofacial implant 210 for interaction with the passive pressure sensor 214 to obtain pressure measurements.

In accordance with this embodiment, and as with the prior embodiment, the passive pressure sensor 14 are those disclosed in U.S. Patent Application Publication No. 20200114353, entitled “Low-Cost Microfluidic Sensors with Smart Hydrogel Patterned Arrays Using Electronic Resistive Channel Sensing for Readout”, which is incorporated herein by reference.

While a hydrogel sensor is disclosed above as a passive present sensor for use in conjunction with the disclosed embodiment, it is appreciated other passive sensors may be employed without departing from the spirit of the present invention.

With regard to the clear craniofacial implant assembly 212 it may take a variety of forms. For example, it might be a universal low-profile intercranial assembly as disclosed above and as disclosed in the '328 Publication referenced above. It may also be static craniofacial implant exhibiting desirably lucent characteristics allowing for the handheld ultrasound transducer 222 to interact with the passive pressure sensor 214 so as to obtain pressure readings. In accordance with the present disclosure a static craniofacial implant 212 is disclosed.

As with the prior embodiment, the static craniofacial implant 212 is a prefabricated implant manufactured from clear poly (methyl methacrylate) (PMMA) or any other clear biocompatible material suited for safe use in craniofacial reconstruction. The clear static craniofacial implant 212 is also sonolucent and has a thickness ranging between 3.0 mm-6.5 mm with a mean thickness of 5.4 mm, which is consistent with native bone flap thickness. While clear sonolucent PMMA is disclosed in accordance with a preferred embodiment, it is appreciated other materials, for example, clear sonolucent PEEK, may be used. The static craniofacial implant 212 may also be radiolucent (that is, allowing passage of radio waves without production of echoes that are due to reflection of some of the waves).

As with the prior embodiment, the clear static craniofacial implant 212 may be manufactured in a manner allowing for the transmission of ultrasonic waves as described in U.S. Pat. No. 9,044,195, entitled “IMPLANTABLE SONIC WINDOW” ('195 Patent) which is incorporated herein by reference. In addition, U.S. Pat. No. 9,535,192, entitled “METHOD OF MAKING WAVEGUIDE-LIKE STRUCTURES” ('192 Publication) and U.S. Patent Application Publication No. 2017/0156596, entitled “CRANIAL IMPLANTS FOR LASER IMAGING AND THERAPY” ('596 Publication) both of which are incorporated herein by reference, making waveguide-like structures within optically transparent materials using femtosecond laser pulses wherein the optically transparent materials are expressly used in the manufacture of cranial implants. Further still, Tobias et al. describe an ultrasound window to perform scanned, focused ultrasound hyperthermia treatments of brain tumors. Tobias et al., “ULTRASOUND WINDOW TO PERFORM SCANNED, FOCUSED ULTRASOUND HYPERTHERMIA TREATMENTS OF BRAIN TUMORS,” Med. Phys. 14(2), March/April 1987, 228-234, which is incorporated herein by reference. Further still, Fuller et al., “REAL TIME IMAGING WITH THE SONIC WINDOW: A POCKET-SIZED, C-SCAN, MEDICAL ULTRASOUND DEVICE,” IEEE International Ultrasonics Symposium Proceedings, 2009, 196-199, which is incorporated herein by reference, provides further information regarding sonic windows.

Radiolucency as applied to the present invention allows a clinician to see the anatomy surrounding the static craniofacial implant 212 without “scatter” or interfering artifacts from the implant for diagnosis and follow-up.

The static craniofacial implant 212 may be of the type described in International Patent Application PCT/US2016/030447, filed May 2, 2017, entitled “LOW PROFILE INTERCRANIAL DEVICE” (published as WO 2017/039762), U.S. patent application Ser. No. 15/669,268, filed Aug. 4, 2017, entitled “METHOD FOR MANUFACTURING A LOW-PROFILE INTERCRANIAL DEVICE AND THE LOW-PROFILE INTERCRANIAL DEVICE MANUFACTURED THEREBY” (published as U.S. Patent Application Publication No. 2018/0055640), and U.S. patent application Ser. No. 16/203,357, filed Nov. 28, 2018, entitled “UNIVERSAL LOW-PROFILE INTERCRANIAL ASSEMBLY” (published as U.S. Patent Application Publication No. 2019/0209328, '328 Publication) all of which are incorporated herein by reference.

The static craniofacial implant 212 includes an outer (commonly convex) first surface 212o, an inner (commonly concave) second surface 212i, and a peripheral edge 212p extending between the outer first surface 212o and the inner second surface 212i. The static craniofacial implant 212 is shaped and dimensioned for engagement with the skull of the patient upon implantation in a manner well known to those skilled in the field of neurosurgical procedures.

The static craniofacial implant 212 has a total thickness similar to that of the embodiment described above, that is, and depending on the strength characteristics of the materials used, the static craniofacial implant 212 will have a thickness (with areas of strategic bulking and/or thinning) of around 1 millimeter to 25 millimeters, preferably, 1 millimeter to 12 millimeters.

As briefly discussed above, the static craniofacial implant 212 includes an outer first surface 212o, an inner second surface 212i, and a peripheral edge 212p, and the passive pressure sensor 214 is positioned adjacent the inner surface 212i such that the passive pressure sensor 214 is exposed to the intracranial fluid for the purposes of sensing intracranial pressure. In accordance with a disclosed embodiment, a plurality of the hydrogel pressure sensors 214 are used.

In accordance with one embodiment the static craniofacial implant 212 includes structure for holding the passive pressure sensor 214 in place. For example, the passive pressure sensor 214 is integrally constructed with the static craniofacial implant 212. For example, a cavity 240 is formed adjacent the inner surface 212i of the static craniofacial implant 212 and the passive pressure sensor 214 is positioned within the cavity 240. However, the cavity 240 is exposed to the intracranial environment via a hole 242 formed along the inner surface 212i of the static craniofacial implant 212 that fluidly connects the inner surface 212i and the cavity 240 so that the passive pressure sensor 214 is exposed to the pressure within the intracranial space.

Considering such an embodiment, it is appreciated it would be possible to use a variety of both passive and active pressure sensors.

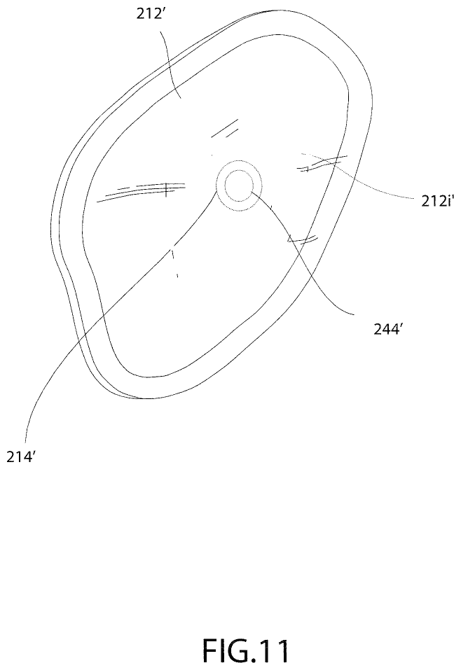

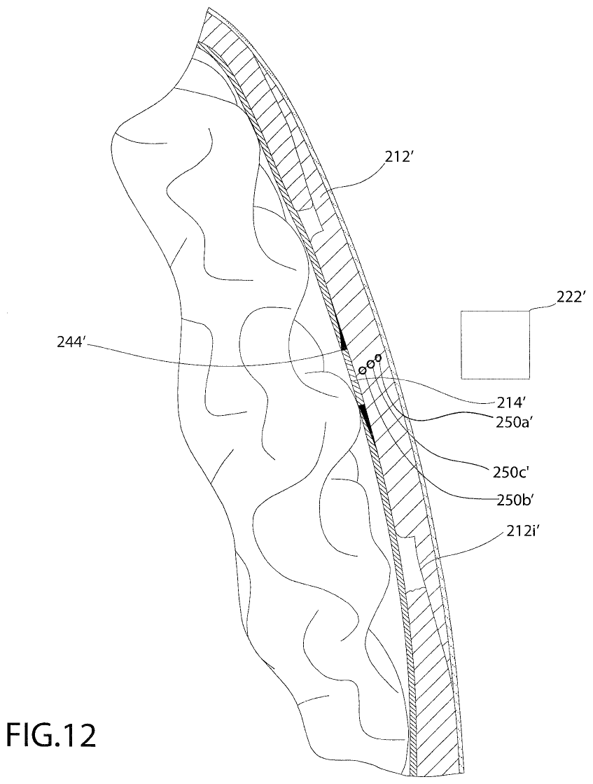

In accordance with another embodiment, the passive pressure sensor 214′ is positioned within a recess 244′ formed along the inner surface 212i′ of the static cranial implant 212′. For example, and with reference to FIGS. 11 and 12, the recess 244′ if formed along the inner surface 212i′ of the static craniofacial implant 212′ and the passive pressure sensor 214′ is positioned within the recess 244′. The passive pressure sensor 214′ is held in place using adhesive or mechanical mounting structures to securely hold the passive pressure sensor 214′ within the recess 244′ and adjacent to the inner surface 212i′ of the static craniofacial implant 212′.

Use of the handheld ultrasound transducer in conjunction with the static cranial implant of this embodiment is enhanced by the integration of alignment mechanisms in the static cranial implant. In particular, the static cranial implant may be constructed with variations in shape designed to control the manner in which light, sound, radio, and other waves pass therethrough. Such variations in shape would be undertaken in a manner similar to the way in which eyeglasses are adjusted for each patient. For example, and with reference to the disclosed embodiment, the curvature of the upper surface differs from the curvature of the lower surface wherein the upper surface has a much larger radius of curvature.

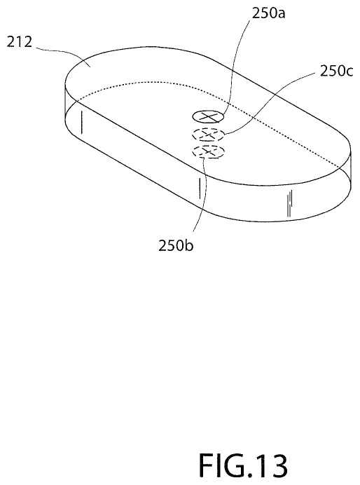

In accordance with another embodiment as shown with reference to FIG. 13, the static cranial implant may be constructed with an alignment feature. In accordance with a disclosed embodiment, the alignment feature includes a series of markings 250a-c, 250a-c′ at different depths within the static cranial implant. For example, an outer first static cranial implant marking 250a, 250a′ and an inner second static cranial implant marking 250b, 250b′ are formed along the outer and inner surfaces 212o, 212i, 212o′, 212i′, respectively, of the static cranial implant 212, 212′. One or more additional interior static cranial implant markings 250c, 250c′ may be formed within the body of the static cranial implant 212, 212′ and in alignment with the outer first static cranial implant marking 250a, 250a′ and inner second lucent disk marking 250b, 250b′. While an outer first static cranial implant marking 250a, 250a′, an inner second static cranial implant marking 250b, 250b′, and at least one additional interior static cranial implant marking 250c, 250c′ are disclosed herein, it is appreciated various combinations of markings may be used within the spirit of the present invention.

The outer first static cranial implant marking 250a, 250a′, the inner second static cranial implant marking 250b, 250b′, and the plurality of additional interior static cranial implant markings 250c, 250c′ are aligned such that when an ultrasound transducer 222, 222′ is properly aligned with the markings, the sound waves will be directed to the proper location within the cranium. Similarly, when one looks through the static cranial implant 212, 212′ and the outer first static cranial implant marking 250a, 250a′, the inner second static cranial implant marking 250b, 250b′, and the at least one additional interior static cranial implant markings 250c, 250c′ merge into a single location identifying image (for example, crosshairs or circles), a specific brain anatomy (or other structural element upon the surface of the brain) is identified by the single location identifying image. When the specific brain anatomy identified by the single location identifying image changes over time, the surgeon will know that something has shifted and will take appropriate action.

While the preferred embodiments have been shown and described, it will be understood that there is no intent to limit the invention by such disclosure, but rather, is intended to cover all modifications and alternate constructions falling within the spirit and scope of the invention.

1. A craniofacial implant, comprising:
a craniofacial implant body; and
a passive pressure sensor;
wherein the craniofacial implant body permits measurement of the passive pressure sensor via externally applied stimuli passing through the craniofacial implant body.

2. The craniofacial implant according to claim 1, wherein the craniofacial implant body is sonolucent.

3. The craniofacial implant according to claim 2, wherein the externally applied stimuli is ultrasound.

4. The craniofacial implant according to claim 1, wherein the craniofacial implant body includes an outer surface, an inner surface, and a peripheral edge shaped and dimensioned for engagement with a skull of a patient upon implantation, and the passive pressure sensor is positioned adjacent the inner surface such that the passive pressure sensor is exposed to intracranial fluid for sensing intracranial pressure.

5. The craniofacial implant according to claim 4, wherein the craniofacial implant body is sonolucent and the externally applied stimuli is ultrasound.

6. The craniofacial implant according to claim 4, wherein the craniofacial implant body includes at least one cavity accommodating displacement of the passive pressure sensor.

7. The craniofacial implant according to claim 4, wherein the craniofacial implant body includes structure holding the passive pressure sensor in place.

8. The craniofacial implant according to claim 4, wherein a recess is formed along the inner surface of the craniofacial implant body and the passive pressure sensor is positioned within the recess.

9. The craniofacial implant according to claim 1, wherein the craniofacial implant body is comprised of clear PMMA (Poly(methyl methacrylate).

10. The craniofacial implant according to claim 1, wherein the craniofacial implant body is sonolucent and the craniofacial implant body has low sound loss permitting measurement of the passive pressure sensor with ultrasonic imaging.

11. The craniofacial implant according to claim 1, wherein the craniofacial implant body is sonolucent and an ultrasound transducer is calibrated or initialized with the passive pressure sensor and/or the craniofacial implant body.

12. The craniofacial implant according to claim 1, wherein the craniofacial implant body is sonolucent and the craniofacial implant further includes an ultrasound transducer transmitting sound waves for interaction with the passive pressure sensor in a predetermined manner based upon physical characteristics of the passive pressure sensor.

13. The craniofacial implant according to claim 1, further including a plurality of a passive pressure sensors.

14. The craniofacial implant according to claim 1, wherein the passive pressure sensor is integrally constructed with the craniofacial implant body.

15. The craniofacial implant according to claim 1, further including a mounting plate in which the craniofacial implant body is selectively positioned.

16. The craniofacial implant according to claim 15, wherein the mounting plate include a hollowed-out center aperture shaped and dimensioned for placement and mounting of the craniofacial implant body therein.

17. The craniofacial implant according to claim 15, wherein the mounting plate comprises PMMA (Poly(methyl methacrylate)), PEEK (Polyether ether ketone), PEKK (Polyetherketoneketone), porous polyethylene, and/or other tissue-engineered constructs.

18. The craniofacial implant according to claim 1, wherein the craniofacial implant body is sonolucent and the craniofacial implant body includes an alignment feature aiding alignment with an external ultrasound transducer.
